(19) United States
(12) Patent Application Publication
(10) Pub. No.: US 2023/0337895 A1
(43) Pub. Date: Oct. 26, 2023

(54) COLONOSCOPY PROBE DEVICE

(71) Applicant: Worcester Polytechnic Institute, Worcester, MA (US)

(72) Inventors: Ahmet Sabuncu, Shrewsbury, MA (US); Cagdas D. Onal, Natick, MA (US); Mary Decelles, Pittsfield, MA (US); Hannah Lindsey, Cincinnati, OH (US)

(21) Appl. No.: 18/138,049

(22) Filed: Apr. 22, 2023

Related U.S. Application Data
(60) Provisional application No. 63/333,646, filed on Apr. 22, 2022.

(51) Int. Cl. A61B 1/00; A61B 18/14

(52) U.S. Cl. CPC A61B 1/00087; A61B 18/1492; A61B 2018/005

(57) ABSTRACT

A colonoscopy screening device includes an elongated flexible probe having a proximal end and a distal end, the distal end adapted for rectal insertion, and an electrode at the distal end and adapted for contact with a suspect tissue growth. A measurement circuit connected to the electrode for measuring an electrical characteristic of the suspect tissue growth based on delivery of an electrical signal from the electrode. A pair of electrodes in an opposed orientation allows an actuator to drawing at least one of the electrodes towards the other electrode for engaging the suspect tissue growth for passing the electrical signal through the suspect tissue growth. Measurement of the resulting signal determines bioelectrical properties of the growth for determination of possible malignancy.

### RELATED APPLICATIONS

This patent application claims the benefit under 35 U.S.C. § 119(e) of U.S. Provisional Patent App. No. 63/333,646, filed Apr. 22, 2022, entitled “COLONOSCOPY PROBE DEVICE,” incorporated herein by reference in entirety.

### BACKGROUND

Colonoscopy procedures are widely employed as preventative measures against colon cancers by allowing detection and removal of precancerous internal growths. There are approximately 1.85 million new cases of colorectal cancer (CRC) per year, and CRC accounts for about 8% of all cancer deaths. CRC arises from growths on the inner lining of the colon called polyps. While hyperplastic polyps are non cancerous, adenomatous polyps could be precursors to cancer. Detection and identification of polyps during regular screening are crucial in the treatment and prevention of CRC.

### SUMMARY

A colonoscopy probe identifies troublesome polyps at the time of screening via electrical measurements. This probe includes a snare tool that can fit within an instrument channel of a typical colonoscopy probe. This snare tool has embedded electrodes to enable electrical resistance measurements. It has been shown that the electrical resistance of polyps decreases as a function of neoplastic progression. The tool assists colonoscopy practitioners with polyp identification by measuring bioelectrical properties of the polyp's tissue in situ.

Configurations herein are based, in part, on the observation that colon screening via colonoscopy is an effective procedure for identifying and removing suspect tissue growth such as polyps from the human colon. Unfortunately, conventional approaches to colonoscopies suffer from the shortcoming that identified growths cannot be analyzed in situ, but rather need to be excised and analyzed by a subsequent laboratory biopsy process. In particular, the laboratory testing is needed to determine if an extracted polyp is hyperplastic or adenomatous.

Colonoscopies have a high initial cost, but by pinpointing where the cost is concentrated can help mitigate cost. The bulk of the expenses result from the removal and submission of diminutive polyps for further testing, and in some cases, such as when a polyp is hyperplastic, non-pre-cancerous, or noncancerous. The greatest opportunity for reducing costs comes from classifying the type of polyp being observed before pathologic examination occurs. Leaving the polyp in place and examining it in its natural state without any surgical removal will allow medical practitioners to avoid surgical and post-surgery lab costs which often turn out to be unnecessary.

Accordingly, configurations herein substantially overcome the shortcomings of growth classification in colonoscopy procedures by providing a remote, actuated device that can fit within a biopsy channel of a conventional colonoscopy probe. This device provides a mechanism that has attached sensors that will help colonoscopists with polyp classification by qualitatively measuring the bioelectrical properties of the polyp's tissue. These sensors are defined by electrodes that will contact the polyp and take resistance measurements of the observed tissue. Currently, colonoscopists only capture visuals of the polyp or remove the polyp entirely to classify whether it is hyperplastic or adenomatous. The use of bioelectrical properties for classification will provide a more accurate understanding of the polyp than just visual determination. By having two identification methods (visual and bioelectrical) for classifying the polyp, colonoscopists can make the best decision for their patient and potentially eliminate costs and inconveniences associated with unnecessary biopsies.

In an example configuration, a colonoscopy screening device includes an elongated flexible probe having a proximal end and a distal end, the distal end adapted for rectal insertion, and an electrode at the distal end and adapted for contact with a suspect tissue growth. A measurement circuit connects to the electrode for measuring an electrical characteristic of the suspect tissue growth based on delivery of an electrical signal from the electrode. A pair of electrodes in an opposed orientation allows an actuator to draw one of the electrodes towards the other electrode for engaging the suspect tissue growth for passing the electrical signal through the suspect tissue growth. Measurement of the resulting signal determines bioelectrical properties of the growth for determination of possible malignancy.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a schematic diagram of the colonoscopy probe and remediation device as disclosed herein;

FIG. 2 shows a bundling for control and electrical resources of the device of claim 1 adapted for routing in an instrument channel of a colonoscopy probe guide device;

FIG. 3 shows tethered control for the snare loop in the device of FIGS. 1 and 2;

FIG. 4 shows the device of FIG. 3 in cooperation with a colonoscopy probe guide;

FIG. 5 shows the snare loop as in FIGS. 1-4 deployed around a polyp;

FIGS. 6A and 6B show electrical characteristics for computing bioimpedance and bioelectrical properties of a suspect tissue growth; and

FIG. 7 shows an alternate deployment of the electrodes as in FIGS. 1-5.

### DETAILED DESCRIPTION

The description below presents an example of a colonoscopy device for engaging a suspect tissue growth in the colon during the colonoscopy procedure for engaging electrodes and passing an electrical signal or current for determining a resistance associated with the growth, typically a polyp, and based on the resistance, classifying the polyp as benign or needing additional biopsy analysis.

A conventional colonoscopy uses a long, flexible tube or probe (colonoscope) that is inserted into the rectum. A small video camera at the tip of the tube allows the doctor to view the colon interior. While observing polyps within the colon, physicians may determine if tissue samples, or biopsies, should be taken for further observation. The colonoscopy screening device as disclosed herein allows qualitative determinations to be conducted immediately, using the biopsy channel of a colonoscope for passing the colonoscopy screening device, or alternatively as a stand-alone device.

Conventional approaches include Computed Tomographic (CT, CAT) colonoscopies, Narrow Band Imaging, Raman Spectroscopy, Diffuse Reflectance Spectroscopy, and Fecal Immunochemical Testing.

Computed tomographic colonoscopies, or a virtual colonoscopy, is a fast-evolving technology that uses data from computed tomography (CT) which generates both two-dimensional and three-dimensional images of the colon and rectum. It is a minimally invasive method and does not require the administration of intravenous sedatives, analgesia, or recovery time to examine the whole colon.

Narrow Band Imaging (NBI) is an advanced visual CRC detection technique. It uses a tight wavelength, or “narrow-band”, illumination of 415±30 nanometers. This light source is then used to optimize hemoglobin light absorption in order to observe neoplastic lesions in a clearer perspective, based on changes in the micro vessels of the mucosa and submucosa.

Raman Spectroscopy is a CRC screening method that relies on observing the interactions between photons and tissue during light scattering. The Raman Spectroscopy system was designed to have the capability to differentiate between normal tissue, hyperplastic polyps, adenomatous polyps, and adenocarcinoma. An advantage of this method over conventional colonoscopy is its ability to produce high-resolution images of both the cellar and tissue features. Another advantage is the spectral differences between adenomas and hyperplasia are apparent. However, a disadvantage to this method is its slow acquisition time which can take up to several minutes.

Diffuse Reflectance Spectroscopy (DRS) is an optical method for tissue identification based on the biochemical composition, oxygenation, and microstructure of the tissue. This is done by delivering light to the tissue and capturing the light that is reflected.

Fecal Immunochemical Testing (FIT) is a noninvasive test that examines stool samples to observe if there is any hidden blood in the lower intestines, which implies a possible presence of cancer, using antibodies.

Of all these conventional colonoscopy cancer detecting methods, none of them incorporate a bioelectrical component. Most of these methods focus on using a visual approach, but as discussed above, optical and manual imaging techniques present accuracy concerns. It would be beneficial to provide a device that assesses harmful polys to minimize surgical extraction and biopsies to determine this information.

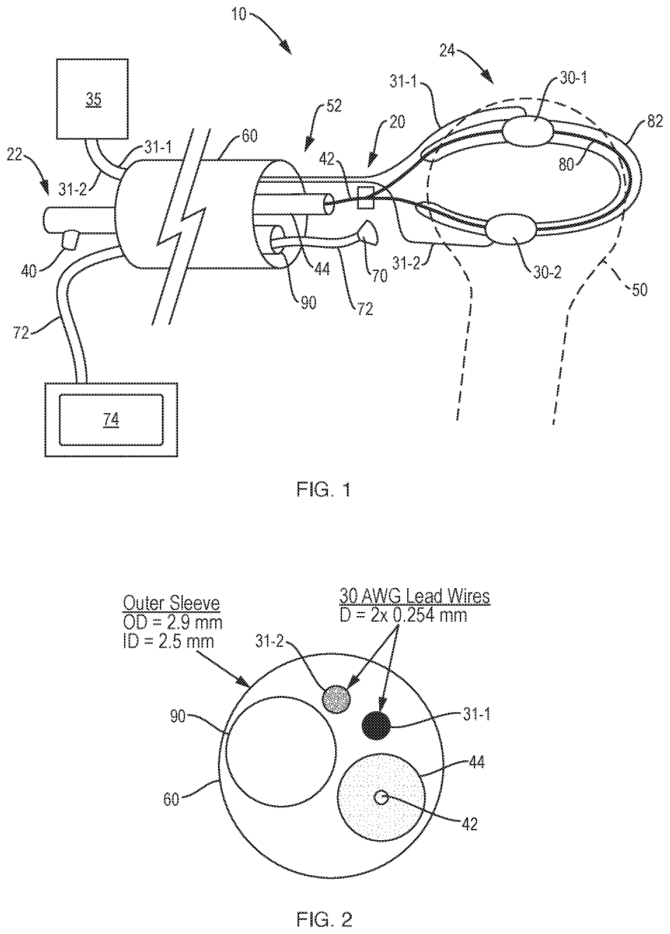

FIG. 1 is a schematic diagram of the colonoscopy probe and remediation device as disclosed herein. Referring to FIG. 1, a colonoscopy screening device 10 (device) includes an elongated flexible probe 20 having a proximal end 22 and a distal end 24, where the distal end 24 is adapted for rectal insertion for direct colon communication. One or more electrodes 30-1 . . . 30-2 (typically 2) at the distal end 24 and adapted for contact with a suspect tissue growth 50. A measurement circuit 35 connected to the electrodes (30 generally) measures an electrical characteristic of the suspect tissue growth 50 based on delivery of an electrical signal from the electrode 30. A respective wire 31-1 . . . 31-2 (31 generally) connects to each electrode. In the example configuration, the electrodes 30 take an opposed orientation at the distal end 24 for flanking the suspect tissue growth 50 (typically a polyp), discussed further below. An actuator 40 allows for drawing at least one of the electrodes towards the other electrode for engaging the suspect tissue growth 50 for passing the electrical signal through the suspect tissue growth 50 and measuring electrical characteristics such as resistance.

The actuator 40 disposes a tethered attachment 42 to the electrodes, where the electrodes 30 are biased in an opposed orientation while the tether 42 is responsive to tension for disposing the electrodes against the bias for closing the electrodes against the suspect tissue growth 50. In an example arrangement, the tether 42 defines a Bowden cable 44 disposed within a probe sleeve 60 (sleeve) along with the wires 31 forming a control bundle 52 within the sleeve. Also included may be a video camera 70 and cable 72 for visual inspection and operational feedback to a visual monitor 74. Alternatively, visual feedback could be provided by a second probe deployed simultaneously.

The electrodes 30 measure a bioelectrical characteristic based on a signal from the measurement circuit 35. The example configuration detects resistance from a small current or voltage signal passed between two electrodes 30 contacting opposed sides of the suspect tissue growth 50. Other bioimpedance measures may be employed. In the example of FIG. 1, a tethered loop 80 defines a snare or similar resilient structure for disposing the electrodes 30 slightly apart for engaging the polyp (suspect tissue growth 50). The tethered loop 80 extends from the distal end for mounting the pair of opposed electrodes 30-1 . . . 30-2. The tethered loop 80 extends from the sleeve or Bowden cable 44 surrounding the tether 42 and attaches to the loop 80 between the electrodes 30.

The tether 42 is responsive to tensioned actuation, or retraction, for drawing the loop at least partially into the sleeve 44 for compressing the loop 80 to draw the electrodes 30 together against the suspect tissue growth 50. When the actuator 40 traverses backward at the proximate end 22, the loop 80, and hence the electrodes 30, close against the polyp at or around a 180 degree opposed position. Diametrical opposition is not required, and depending on the diameter of the polyp, contact locations may vary. Resistance is still measurable, as discussed below, based on electrical characteristics, often ion or NaCl concentration in the cells of the suspect growth 50.

Emanating from the measurement circuit 35 are a respective wire 31 terminating in the respective electrodes 30. The loop 80 and retraction tether 42 may be metal, and hence conductive, particularly when a Bowden cable 42 is employed. Accordingly, an insulative coating 82 on the loop provides an electrical isolation between the electrodes 30 for preventing electrical communication over the loop. Retraction of the tether 42 into the sleeve 44 then engages the pair of electrodes 30 on opposed sides of a surface of the suspect tissue growth 50, forming an electrical continuity from the measurement circuit, down the wires 31 to the electrodes 30 and across the suspect tissue growth 50 for defining an electrical resistance of the polyp measurable by the measurement circuit 35.

FIG. 2 shows a bundling in the probe 20 for control and electrical resources of for routing in an instrument channel of a colonoscopy probe guide device. An outer sleeve 60 encloses the tether 42 and the respective wires 31, such that the outer sleeve 60 is adapted for passage in a biopsy channel of a colonoscope. The diameter of a biopsy channel ranges from 3.2 mm to 3.6 mm, so the outer sleeve 60 preferably does not exceed a 3.0 mm diameter to ensure sufficient clearance within the biopsy channel of a standard colonoscope. Also accommodated is a utility channel 90, which may provide for a video camera cable or wire or a suction source, discussed further below.

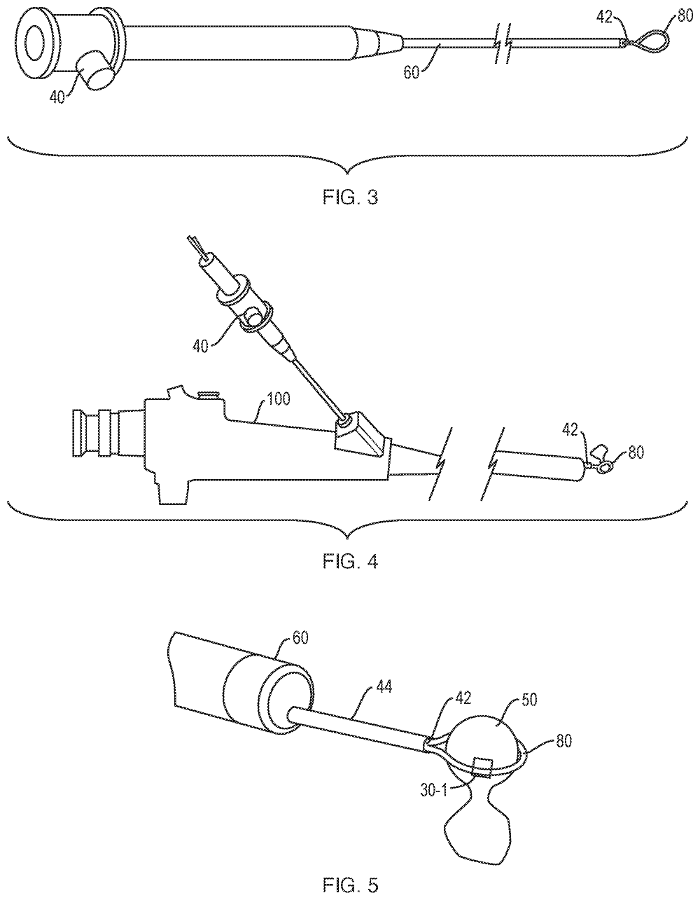

FIG. 3 shows tethered control for the snare loop in the device of FIGS. 1 and 2. Referring to FIGS. 1-3, the tether 42 further comprises a Bowden cable. Movement of the actuator 40 slides the cable 42 for extending the snare loop 80.

FIG. 4 shows the device of FIG. 3 in cooperation with a colonoscopy probe guide 100. Conventional colonoscopy probes employ a handheld guide for delivering a biopsy specimen and a video camera, allowing biopsy extraction based on visual inspection. The claimed approach is retroactively compatible based on a sizing of the outer sleeve 60 having a diameter based on a biopsy channel of a colonoscope. The outer sleeve 60 therefore encloses the tether 42 and the respective wires 30 where the outer sleeve 60 is adapted for passage in a biopsy channel of a conventional colonoscope. In a typical arrangement, the outer sleeve 60 has a diameter between 3.0-3.6 mm.

FIG. 5 shows the snare loop as in FIGS. 1-4 deployed around a polyp. Referring to FIGS. 1-5, the polyp is a typical type of suspect tissue growth 50 that the device 10 targets. In operation, the method for colon screening includes extending an electrical probe into a colorectal diagnostic region, and disposing the electrodes 30 in contact with the suspect tissue growth 50. The electrodes 30 contact the polyp for measuring a resistance from an electrical signal emanating from the electrode 30, to determine, based on the resistance, whether the suspect tissue growth 50 should be removed.

The loop 80 is formed at the end of the tether 42 by applying an electrically insulative coating to a tethered loop 80, and attaching the electrodes 30 to opposed sides of the tethered loop, such that the electrically insulative coating provides an electrical separation between the electrodes. Retraction of the tether closes the loop to draw the electrodes 30 into contact with the suspect tissue growth 50.

The operation of the loop 80 serves to separate the pair of electrodes 30-1 . . . 30-2 by a distance based on a size of the suspect tissue growth as the loop is sufficiently loose to be slid over the polyp. Extension of the tether 42 moves the electrodes toward the respective opposed sides of the suspect tissue growth 50, and tether 42 retraction then closes the electrodes 30 around the respective tissue growth 50 by reducing the distance between the electrodes to achieve electrical contact of the electrodes with the respective opposed sides.

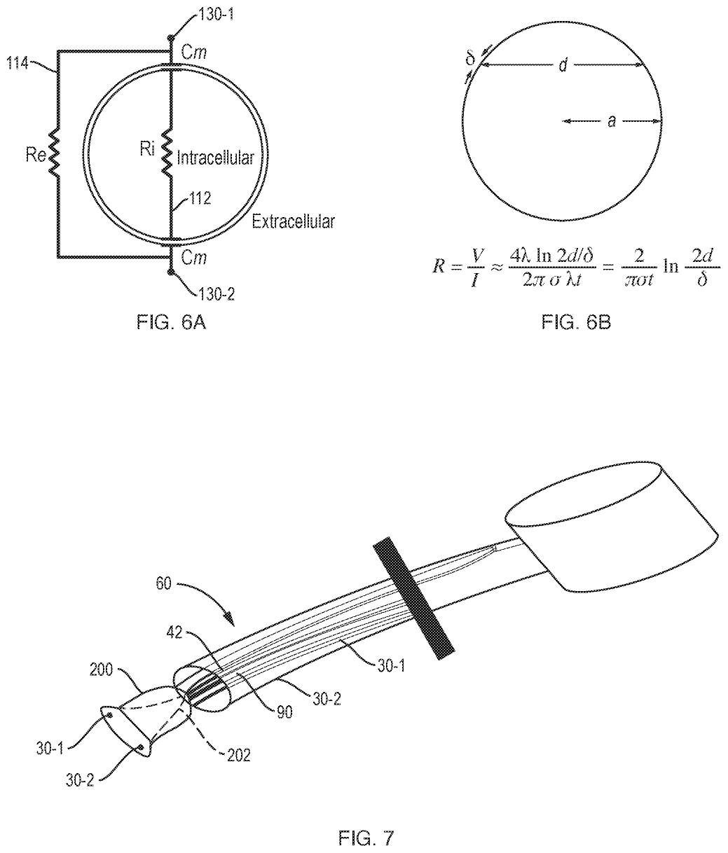

FIGS. 6A and 6B show electrical characteristics for computing bioimpedance and bioelectrical properties of a suspect tissue growth. Upon placement of electrodes on a cell 110, and applying a voltage difference between electrodes 130-1 . . . 130-2, a resistance across 112 the cell is different than a conductor around 114 the cell. Bioimpedance is a cumulative term that describes safe, non-invasive methods that measure the electrical responses to low-level, alternating current into living organisms, and the biophysical models to interpolate body composition from bioelectrical measurements. The basis of bioimpedance is codified in studies of the body's structure and function uses an electrical circuit to describe bioelectrical components of a living cell. This model relates extracellular and intracellular ionic fluids to parallel resistors (Re and Ri) and the cell membrane to a capacitor (Cm).

Bioelectrical impedance analysis (BIA) therefore is a technique based on measuring the resistance or reactance of an alternating current in the human body. Resistance is the opposition to the flow of an alternating or direct current in the body by intracellular fluids, body fluids, and electrolytes behaving as electrical conductors.

FIG. 6B extends the cell model and analysis of FIG. 6A to a multicellular polyp structure defined by a generally round structure with a determinable radius. In the equation shown in FIG. 6B, R represents resistance, which is a value obtained from the multimeter, σ is conductivity which is equal to 1/ρ, or 1 divided by resistivity of the given material, t represents the thickness of contact point, or diameter of the wire, d represents the distance between contact points, and δ represents the width of the contact point.

FIG. 7 shows an alternate deployment of the electrodes as in FIGS. 1-5. Recall that the insulated snare loop of FIGS. 1 and 5 provides a deployment of electrodes on opposing sides of the polyp for measuring a resistance. In FIG. 7, the electrodes are separated by a biasing member 202 for disposing the electrodes 30-1 . . . 30-2 in a biased, opposed arrangement for engaging the polyp. A deformable, concave sleeve 200 around the electrodes 30 has an elastic exterior and connects with a suction tube for contracting the sleeve. The suction tube may reside in the channel 90, and pulls the electrodes against the polyp when the sleeve forms a sealing engagement with the polyp such that a small vacuum contracts the sleeve 200 and compresses the electrodes 30 against the polyp. Other suitable mechanisms for drawing the electrodes 30 together around the polyp or other suspect tissue growth 50 may be pursued.

Those skilled in the art should readily appreciate that the programs and methods defined herein are deliverable to a user processing and rendering device in many forms, including but not limited to a) information permanently stored on non-writeable storage media such as ROM devices, b) information alterably stored on writeable non-transitory storage media such as solid state drives (SSDs) and media, flash drives, floppy disks, magnetic tapes, CDs, RAM devices, and other magnetic and optical media, or c) information conveyed to a computer through communication media, as in an electronic network such as the Internet or telephone modem lines. The operations and methods may be implemented in a software executable object or as a set of encoded instructions for execution by a processor responsive to the instructions, including virtual machines and hypervisor controlled execution environments. Alternatively, the operations and methods disclosed herein may be embodied in whole or in part using hardware components, such as Application Specific Integrated Circuits (ASICs), Field Programmable Gate Arrays (FPGAs), state machines, controllers or other hardware components or devices, or a combination of hardware, software, and firmware components.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A colonoscopy screening device, comprising:
an elongated flexible probe having a proximal end and a distal end, the distal end adapted for rectal insertion;
an electrode at the distal end and adapted for contact with a suspect tissue growth; and
a measurement circuit connected to the electrode for measuring an electrical characteristic of the suspect tissue growth based on delivery of an electrical signal from the electrode.

2. The device of claim 1 further comprising:
a pair of electrodes in an opposed orientation at the distal end; and
an actuator for drawing at least one of the electrodes towards the other electrode for engaging the suspect tissue growth for passing the electrical signal through the suspect tissue growth,

3. The device of claim 2 wherein the actuator includes a tethered attachment to the electrodes, the electrodes biased in an opposed orientation, the tether responsive to tension for disposing the electrodes against the bias for closing the electrodes against the suspect tissue growth.

4. The device of claim 1 further comprising:
a tethered loop extending from the distal end, the tethered loop having a pair of opposed electrodes;
the tethered loop extending from a sleeve, the sleeve surrounding a tether attached to the loop between the electrodes,
the tether responsive to tensioned actuation for drawing the loop at least partially into the sleeve for compressing the loop for drawing the electrodes together against the suspect tissue growth.

5. The device of claim 4 further comprising:
a respective wire terminating in the respective electrodes; and
an insulative coating on the loop, the insulative coating providing an electrical isolation between the electrodes for preventing electrical communication over the loop.

6. The device of claim 5 further comprising an outer sleeve enclosing the tether and the respective wires, the outer sleeve adapted for passage in a biopsy channel of a colonoscope.

7. The device of claim 2 further comprising:
a biasing member for disposing the electrodes in a biased, opposed arrangement;
a deformable, concave sleeve around the electrodes;
a suction tube in fluidic communication with the concave sleeve; and
a vacuum source attached to the suction tube and adapted for reducing a pressure in the concave sleeve, the concave sleeve adapted to contract in response to the reduced pressure upon a sealing engagement with the suspect tissue growth,
the contraction of the sleeve forcing the electrodes against the suspect tissue growth.

8. The device of claim 4 wherein the tether further comprises a Bowden cable, further comprising a pair of wires attached, each wire of the pair of wires attached to a respective electrode, the Bowden cable and the pair of wires enclosed in a bundle within an outer sleeve, the outer sleeve extending from a colonoscope.

9. The device of claim 6 wherein the outer sleeve has a diameter based on a biopsy channel of a colonoscope.

10. The device of claim 6 wherein the outer sleeve has a diameter between 3.0-3.6 mm.

11. A method for colon screening, comprising:
extending an electrical probe into a colorectal diagnostic region;
disposing the electrode in contact with a suspect tissue growth;
measuring a resistance from an electrical signal emanating from the electrode; and
determining, based on the resistance, whether the suspect tissue growth should be removed.

12. The method of claim 11 further comprising:
engaging a pair of electrodes on opposed sides of a surface of the suspect tissue growth, and
measuring the resistance between the electrodes based on cell properties of the suspect tissue growth.

13. The method of claim 11 further comprising:
separating the pair of electrodes by a distance based on a size of the suspect tissue growth;
extending the electrodes adjacent toward the respective opposed sides of the suspect tissue growth; and
closing the electrodes around the respective tissue growth by reducing the distance for electrical contact of the electrodes with the respective opposed sides.

15. The method of claim 12 further comprising:
applying an electrically insulative coating to a tethered loop;
attaching the electrodes to opposed sides of the tethered loop, the electrically insulative coating providing an electrical separation between the electrodes; and
retracting the tether to close the loop and draw the electrodes into contact with the suspect tissue growth.

16. The method of claim 15 wherein the tether further comprises a Bowden cable.
